(19) United States
(12) Patent Application Publication
(10) Pub. No.: US 2023/0052157 A1
(43) Pub. Date: Feb. 16, 2023

(54) METHOD FOR OBTAINING NUCLEIC ACID FOR SEQUENCING

(71) Applicant: Achilles Therapeutics UK Limited, London (GB)

(72) Inventors: Gareth Wilson, London (GB); Pablo Becker, London (GB); Sonia Ventura, London (GB); Maximilian Prinz zu Salm-Horstmar, London (GB)

(21) Appl. No.: 17/784,324

(22) PCT Filed: Dec. 11, 2020
(86) PCT No.: PCT/GB2020/053201

§ 371 Date: Jun. 10, 2022

(30) Foreign Application Priority Data
Dec. 12, 2019 (GB) 1918290.6
Sep. 29, 2020 (GB) 2015380.5

(51) Int. Cl. C12Q 1/6886

(52) U.S. Cl. CPC C12Q 1/6886; C12Q 1/6869

(57) ABSTRACT

The present invention provides a method for obtaining tumour nucleic acid for sequencing, comprising providing a medium containing tumour cells shed from a solid tumour sample into the medium ex vivo and/or released during mechanical disruption of a solid tumour sample and extracting nucleic acid from the shed and/or released tumour cells tumour cells.

### FIELD OF THE INVENTION

The present invention relates to a method for obtaining nucleic acid from tumours from patients, in particular for the purpose of sequencing said nucleic acid. Said method comprises providing a medium containing tumour cells which have been shed from a tumour sample into the medium ex vivo, and extracting nucleic acid from the shed tumour cells. The sequence information obtained from said method may be used to identify clonal neoantigens which may be targeted in the treatment of a tumour. The invention also relates to methods for expanding T cell populations specific for said clonal neoantigens which may be used for treating cancer in a patient.

### BACKGROUND

Genetic instability of tumour cells often leads to the occurrence of a large number of mutations, and expression of non-synonymous mutations can produce tumour-specific antigens called neoantigens. Neoantigens are non-autologous proteins with individual specificity, which are generated by non-synonymous mutations in the tumour cell genome. Neoantigens are highly immunogenic as they are not expressed in normal tissues. They can activate CD4+ and CD8+ T cells to generate immune response and so are ideal targets for tumour immunotherapy. The development of bioinformatics technology has accelerated the identification of neoantigens. The combination of different algorithms to identify and predict the affinity of neoantigens to major histocompatibility complexes (MHCs) or the immunogenicity of neoantigens is mainly based on whole-exome sequencing technology.

In order to target a tumour neoantigen, the neoantigen must firstly be identified, which involves sequencing of nucleic acid from a tumour. This necessarily involves obtaining nucleic acid from the tumour of interest.

It is advantageous to obtain said nucleic acid for sequencing from as small amount of tumour sample as possible, leaving the remaining tumour available for further pathological analysis or the production of therapeutic T cell populations that may target tumour neoantigens.

There is therefore a need in the art for a means of efficiently obtaining tumour nucleic acid from a sample of tumour. It is advantageous to be able to obtain sufficient amounts of nucleic acid from a tumour sample for sequencing purposes wherein said nucleic acid is of sufficient amount and quality to enable the identification of neoantigens, particularly clonal neoantigens.

### BRIEF DESCRIPTION OF THE INVENTION

The present inventors have surprisingly found that cells which have been shed from a solid tumour sample into a medium, for example following tumour resection, provide sufficient nucleic acid of suitable quality to enable sequencing of the nucleic acid, for example to identify neoantigens that may be targeted in the treatment of cancer. The tumour cells that are shed into the medium may be representative of the tumour as a whole, and may provide sufficient sequence information to enable the detection of the vast majority of ubiquitous, or clonal, mutations within the tumour.

Accordingly, the present invention provides a method for obtaining tumour nucleic acid for sequencing, comprising providing a medium containing tumour cells shed from a solid tumour sample and/or released during mechanical disruption of at least part of the solid tumour sample, and extracting nucleic acid from the shed and/or released tumour cells.

In one aspect the method comprises the following steps:

(a) providing a medium containing tumour cells shed from a solid tumour sample and/or released during mechanical disruption of at least part of the solid tumour sample;

(b) isolating the shed and/or released tumour cells from the medium; and

(c) extracting nucleic acid from the shed and/or released tumour cells.

In one aspect the medium contains tumour cells which have been shed from a solid tumour sample directly into the medium ex vivo or in vitro.

In one aspect the solid tumour is selected from non-small cell lung cancer (NSCLC), melanoma, renal cancer, bladder cancer, head and neck cancer, and breast cancer.

In one aspect the method comprises the step of sequencing nucleic acid extracted from the shed and/or released tumour cells. In one aspect the sequence information generated is suitable for the identification of a clonal neoantigen(s) from the tumour. In one aspect the method comprises the step of identifying a clonal neoantigen(s) from the tumour.

In one aspect the method may further comprise isolating tumour infiltrating lymphocytes (TIL) from at least part of the solid tumour sample. The TIL may be selectively expanded to produce a population of clonal neoantigen-specific T cells (cNeT).

In one aspect the method may further comprise the step of mechanically disrupting at least part of the solid tumour sample and extracting nucleic acid from tumour cells released during the mechanical disruption. In one aspect the method does not comprise a step of enzymatically disrupting the solid tumour sample prior to the extraction of nucleic acid for sequencing.

In one aspect the method may comprise the step of removing non-tumour cells by negative selection prior to the extraction of nucleic acid for sequencing, for example by immunomagnetic negative selection.

The invention as described herein also encompass the use of tumour cells which have been shed from a solid tumour sample into a medium ex vivo to provide tumour nucleic acid for sequencing.

The invention also provides a method for selectively expanding a T cell population for use in the treatment of cancer in a subject, the method comprising the steps of:
(a) providing medium containing tumour cells shed from a solid tumour sample and/or released during mechanical disruption of at least part of the tumour sample;
(b) extracting nucleic acid from the shed and/or released tumour cells;
(c) sequencing nucleic acid extracted from the shed and/or released tumour cells;
(d) identifying a clonal neoantigen from the tumour using the sequence information obtained in step (c);
(e) isolating tumour infiltrating lymphocytes (TIL) from at least part of said solid tumour sample; and
(f) co-culturing the TIL with a peptide comprising the clonal neoantigen identified in step (d) and an antigen presenting cell.

In another aspect the present invention provides a method for obtaining tumour nucleic acid for sequencing, comprising providing a medium containing tumour cells shed or released from at least part of a tumour sample during mechanical disruption of the at least part of the tumour sample, and preferably extracting nucleic acid from said tumour cells.

In one aspect the tumour nucleic acid extracted from the tumour cells shed from a solid tumour sample may be combined with nucleic acid extracted from tumour cells released during mechanical disruption of at least part of the tumour sample. In one aspect the combined extracted nucleic acid may be sequenced.

### DESCRIPTION OF THE FIGURES

FIG. 1—Gating strategy for identifying tumour cells within a heterogeneous cell suspension. Based on sequential exclusion of known contaminating cells (leukocytes, endothelial cells and fibroblasts).

FIG. 2—Enzymatically dissociated tumour fragments have low tumour cell yield. A. Tumour cell frequencies in cell suspensions obtained by enzymatic dissociation were estimated by flow cytometry. B. The estimated yield of tumour cells for each sample was calculated by multiplying the tumour cell frequency by the total number of cells obtained in the cell suspension. The tumour cell yield was normalised to the size of a typical tumour fragment received for processing (0.1 gram). The dotted line highlights the 1 million cell threshold. Viable cell counts were performed using a haemocytometer and trypan blue staining. The average of each group is indicated as geometric mean. N=5-8/group.

FIG. 3—Transport media contains good numbers of tumour cells. A. Tumour cell frequencies in cell suspensions obtained from transport media were estimated by flow cytometry. B. The estimated yield of tumour cells for each sample was calculated by multiplying the tumour cell frequency by the total number of cells obtained in the cell suspension. The estimated tumour cell yield was normalised to the respective total tumour weight excised and carried in the media. The dotted line highlights the 1 million cell threshold. C. Viable cell counts were performed using a haemocytometer and trypan blue staining. Data is shown as percentage of viable cells in the cell suspension. The average of each group is indicated as geometric mean. N=4/group.

FIG. 4—Tumour cell numbers shed into the transport media. A. The estimated yield of tumour cells for each sample was calculated by multiplying the tumour cell frequency by the total number of cells obtained in the cell suspension. The estimated tumour cell yield was normalised to the respective total tumour weight excised and carried in the media. The dotted line highlights the 1 million cell threshold. B. Viable cell counts were performed using a haemocytometer and trypan blue staining. Data is shown as percentage of viable cells in the cell suspension. N=4/group.

FIG. 5—TM and CM tumour cell parameters are similar, independently of tumour type. A. Tumour cell frequencies in cell suspensions obtained from transport media (TM) and cutting media (CM) were estimated by flow cytometry. B. The estimated yield of tumour cells for each sample was calculated by multiplying the tumour cell frequency by the total number of cells obtained in the cell suspension. The estimated tumour cell yield was normalised to the respective total tumour weight excised and carried in the media. The dotted line highlights the 1 million cell threshold.

FIG. 6—Pooling TM and CM provides more consistent cell numbers across different tumour types. A. Tumour cell frequencies in cell suspensions obtained from transport media and cutting media were estimated by flow cytometry. B. The estimated yield of tumour cells for each sample was calculated by multiplying the tumour cell frequency by the total number of cells obtained in the cell suspension. The estimated tumour cell yield was normalised to the respective total tumour weight excised and carried in the media. The dotted line highlights the 1 million cell threshold.

FIG. 7—Tumour cell suspensions obtained from either enzymatic or mechanical dissociation are infiltrated by cells of non-tumour origin. Cell frequency for the most common nucleated contaminating cells (leukocytes, endothelial cells and fibroblasts) was estimated by flow cytometry and data is shown as percentage of contaminating cells in single, live cells. ED—Enzymatic dissociation; MD—mechanical dissociation (TM+CM); N=5-12/group.

FIG. 8—Low frequency tumour cell suspensions can be successfully enriched by immunomagnetic negative selection. A. Tumour cell frequencies in either non-purified or purified fractions were estimated by flow cytometry, and data is shown as percentage of tumour cells in single, live cells. Frequencies in cell suspensions obtained from transport media were estimated by flow cytometry. B. The estimated yield of tumour cells for each sample was calculated by multiplying the tumour cell frequency by the total number of cells obtained in the cell suspension. The estimated tumour cell yield was normalised to the respective total tumour weight excised and carried in the media. The dotted line highlights the 1 million cell threshold. C. Recovery rates for each one of the tested purification strategies were calculated as: Recovery [%]=100×([No. of cells in enriched fraction]×[Tumour cell frequency in enriched fraction])/([No. of cells in original fraction]×[Tumour cell frequency in original fraction]). D. Viable cell counts in either non-purified or purified fractions were obtained using a haemocytometer and trypan blue staining. Data is shown as percentage of viable cells in the sample. N=15-25/group.

FIG. 9—Orthogonal validation from whole exome sequencing illustrates successful enrichment of tumour cells in NSCLC and Melanoma samples. Tumour cell frequencies in non-purified and purified samples were estimated computationally by ASCAT for a NSCLC patient “Patient 1” (A) and a melanoma patient “Patient 2” (B). The plots show the increase in tumour cell content in the purified samples. Variant allele frequencies for the somatic mutations identified in the non-purified and purified samples from patients “Patient 1” (C) and “Patient 2” (D) were grouped according to their prevalence in the matched multi-region dataset. Frequencies are shown to be higher in the purified compared to the non-purified samples and the frequencies of ubiquitous/clonal and shared mutations are higher than those of private mutations, reflecting the likely abundance of cells carrying the mutation in the primary tumour.

FIG. 10—Positive percent agreement of clonal mutations identified in the primary multi-region dataset and subsequently detected in the matched TM and CM samples. The percentage of mutations classed as ubiquitous or clonal in the fresh frozen multi-regional whole exome dataset and detected in the non-purified and purified samples for patients (A)+(C) “Patient 1” (n=41) and (B)+(D) “Patient 2”.

FIG. 11—Positive percent agreement of mutations identified in the primary multi-region dataset and subsequently detected in the matched TM sample. The percentage of mutations classed as ubiquitous (or clonal), shared and private in the fresh frozen multi-regional whole exome dataset and detected in the purified TM

FIG. 12—Positive percent agreement of mutations identified in the primary multi-region dataset and subsequently detected in the matched CM sample. The percentage of mutations classed as ubiquitous (or clonal), shared and private in the fresh frozen multi-regional whole exome dataset and detected in the purified CM

FIG. 13—Positive percent agreement of mutations identified in the primary multi-region dataset and subsequently detected in the matched TM sample obtained from a

Head and Neck Squamous Carcinoma. The percentage of mutations classed as ubiquitous (or clonal), shared and private in the fresh frozen multi-regional whole exome dataset and detected in the purified TM

FIG. 14—Positive percent agreement of mutations identified in the primary multi-region dataset and subsequently detected in the matched CM sample obtained from a Head and Neck Squamous Carcinoma. The percentage of mutations classed as ubiquitous (or clonal), shared and private in the fresh frozen multi-regional whole exome dataset and detected in the purified CM

### DETAILED DESCRIPTION OF THE INVENTION

As described herein, the present invention provides a method for obtaining tumour nucleic acid suitable for sequencing, comprising providing a medium containing tumour cells shed from a solid tumour sample and extracting nucleic acid from the shed tumour cells.

Also provided is a method for sequencing nucleic acid from a solid tumour, wherein said method comprises the steps of:

(i) providing a medium containing tumour cells shed from a solid tumour sample; and

(ii) extracting nucleic acid from the shed tumour cells. The term “shed” is intended to describe tumour cells which are detached from a tumour. As such, shed tumour cells are free in the medium and are not directly physically connected to the solid tumour sample. The term “shed” is intended to describe tumour cells which are shed passively into the medium, i.e. they have not been actively dissociated from the solid tumour sample. These tumour cells will typically be shed from the exterior surfaces of the solid tumour sample into the medium.

The shed tumour cells may have passively separated from the tumour sample during a period of retention in the medium.

In one aspect the medium contains tumour cells which have been shed from a solid tumour sample directly into the medium ex vivo.

In one aspect the tumour sample may be retained in the medium as described herein for a period of time, and shed tumour cells are those which dissociate from the tumour sample during this period.

Shed tumour cells are distinct from circulating tumour cells which are released from a primary tumour into the blood in vivo. The shed tumour cells utilised in the methods of the present invention are directly shed from a tumour sample into a medium during retention of the tumour sample in said medium in vitro or ex vivo.

In one aspect of the invention the method is an in vitro or ex vivo method.

In one aspect of the invention the tumour sample is not cultured in vitro or ex vivo prior to extraction of the nucleic acid.

In one aspect the tumour sample is not an explant culture. In one aspect the shed cells are not shed during in vitro or ex vivo culture of a tumour explant. In one aspect the tumour sample is not in vitro or ex vivo cultured tumour cells. In one aspect the shed cells are not shed during in vitro or ex vivo culture of tumour cells.

In one aspect the shed cells are not collected or isolated from culture medium, i.e. medium in which cells or tumour explant have been cultured (for example supernatant medium in a culture vessel). In one aspect the cells are not collected or isolated from a culture vessel.

In one aspect the solid tumour sample itself is not used to extract nucleic acid. That is to say, the nucleic acid is not extracted from the intact solid tumour sample. In one aspect the solid tumour sample is not used to extract nucleic acid for sequencing (as such, the liquid medium component only is used for nucleic acid extraction).

In one aspect there is no disruption of the solid tumour sample by enzymatic or other non-mechanical means prior to nucleic acid extraction.

In another aspect there is no disruption of the solid tumour sample by mechanical means prior to nucleic acid extraction. In one aspect the method does not comprise the step of mechanically disrupting at least part of the solid tumour sample and extracting nucleic acid from tumour cells released during the mechanical disruption. As such, nucleic acid is extracted only from tumour cells shed into the medium in which the solid tumour sample has been transported and/or retained.

### Medium

Any suitable medium for transporting, retaining or storing a solid tumour sample may be used according to the present invention. The medium may be any suitable medium that maintains cell viability. For example, in one aspect the medium may be HypoThermosol® biopreservation medium (BioLife Solutions).

Other suitable media include, but are not limited to, Dulbecco's Modified Eagle's Medium (DMEM), Ham's F10 medium, Ham's F12 medium, Advanced DMEM, Advanced DMEM/F12, minimal essential medium, DMEM/F-12, DMEM/F-15, Liebovitz L-15, RPMI 1640, Iscove's modified Dulbecco's media (IMDM), OPTI-MEM SFM (Invitrogen Inc.), N2B27, MEF-CM or a combination thereof. In one aspect the medium may be phosphate buffered saline (PBS).

In one aspect the medium may comprise serum, for example fetal calf serum. In one aspect the medium may comprise 1%, 2%, 3%, 4%, 5%, 6%, 7%, 8%, 9%, 10%, 11%, 12%, 13%, 14%, 15%, 16%, 17%, 18%, 19% or 20% fetal calf serum.

In one aspect the medium may comprise an antibiotic and/or fungicide. In one aspect the medium may comprise other supplements, such as glutamine or HEPES, or any other supplement that assists in maintaining cell viability. Such supplements will be known to one of skill in the art.

The term “medium” as used herein is not intended to encompass a biological sample from a subject. In one aspect the medium is not a biological sample from a subject, for example the medium is not blood or a blood fraction, such as serum or plasma or peripheral blood mononuclear cells. In one aspect the medium is not saliva, lymph, pleural fluid, ascites, or cerebrospinal fluid.

According to methods of the invention as described herein, the solid tumour sample may be, or have been, retained in the medium as described herein for a period of at least about 5 minutes, 10 minutes, 15 minutes, 30 minutes, 60 minutes/1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours or 12 hours or more prior to a step of extracting nucleic acid from the shed tumour cells.

In one aspect the solid tumour sample may be, or have been, retained in the medium as described herein for a period of at least about 1 hour.

The solid tumour sample may be, or have been, retained in the medium as described herein for a period of at least about 3.5 hours.

The transport medium may be used to transport the tumour sample from the operating theatre following surgical resection of the tumour.

### Sample

As referred to herein, a “tumour sample” refers to a sample deriving or obtained from a tumour. The tumour according to the present invention is a solid tumour.

Isolation of biopsies and samples from tumours is common practice in the art and may be performed according to any suitable method, and such methods will be known to one skilled in the art.

The tumour sample may be a primary tumour sample, tumour-associated lymph node sample or sample from a metastatic site from the subject.

Tumour samples and non-cancerous samples can be obtained according to any method known in the art. For example, solid tumour samples can be obtained from cancer patients that have undergone resection, or they can be obtained by extraction using a hypodermic needle, punch biopsy, microdissection, or laser capture. Control (non-cancerous) samples if needed may be obtained, for example, from a blood sample or normal tissue adjacent to the tumour.

### Mechanical Disruption

In one aspect of the invention the method may comprise the step of mechanically disrupting at least part of the solid tumour sample and extracting nucleic acid from tumour cells released during the mechanical disruption.

By “released” is intended to refer to cells that have become dissociated from the solid tumour sample during the mechanical disruption, for example cells internal to the solid tumour sample.

In another aspect, the present invention provides a method for obtaining tumour nucleic acid for sequencing, comprising providing a medium containing tumour cells released during mechanical disruption of at least part of the tumour sample.

The invention also provides a method for obtaining tumour nucleic acid for sequencing, comprising providing a medium containing tumour cells released from at least part of a tumour sample during mechanical disruption of the at least part of the tumour sample and extracting nucleic acid from said tumour cells.

Also provided is a method for sequencing nucleic acid from a solid tumour, wherein said method comprises the steps of:

(i) providing a medium containing tumour cells released from at least part of a tumour sample during mechanical disruption of the at least part of the tumour sample; and

(ii) extracting nucleic acid from the shed tumour cells.

In one aspect neither said tumour sample nor said released cells are cultured ex vivo prior to extraction of said nucleic acid. In one aspect there is no disruption of the solid tumour sample by enzymatic or other non-mechanical means prior to nucleic acid extraction.

The method according to the invention may comprise the following steps:

(a) providing a medium containing tumour cells released from a solid tumour sample;

(b) isolating the released tumour cells from the medium; and

(c) extracting nucleic acid from said tumour cells.

In one aspect the released cells are not collected or isolated from culture medium, i.e. medium in which cells or tumour explant have been cultured (for example supernatant medium in a culture vessel). In one aspect the cells are not collected or isolated from a culture vessel.

Said mechanical disruption may be performed by methods known in the art, for example mincing or dissection of the tumour sample.

The medium into which tumour cells are released during mechanical disruption may be referred to as a cutting medium. The cutting medium may be the medium in which at least part of the tumour sample is processed (see Example 2, for example) or the medium used to clean and/or rinse or flush out the equipment used to cut, mince and/or dissect the tumour sample.

The mechanical disruption applied in the present invention may cut the at least part of the tumour sample into small pieces, for example about 0.5 to 10 mm³, about 1 to 6 mm³ or about 1 to 3 mm³. Preferably, the mechanical disruption may cut the at least part of the tumour sample into pieces of about 1 to 3 mm³.

The mechanical disruption may cut the at least part of the tumour sample into pieces of at least 0.5, 1, 1.5, 2, 2.5, 3, 5, 7 or 9 mm³.

The mechanical disruption may cut the at least part of the tumour sample into pieces of at least 1, 1.5, 2 or 2.5 mm³.

The present mechanical disruption or cutting is distinct to a homogenisation step, which typically comprises processing a sample into smaller pieces than described herein. For example, a homogenised sample may contain tissue that has been dissociated into individual cells or small clusters of cells (e.g. fewer than 1000 cells) and may be referred to as a liquid or liquefied sample based on its ability to flow.

In one aspect the method does not comprise a step of enzymatically disrupting the solid tumour sample prior to the extraction of nucleic acid for sequencing.

In one aspect the method does not comprise a step of homogenising at least part of the tumour sample prior to the extraction of nucleic acid for sequencing. Said homogenisation may be a mechanical or enzymatic disruption step.

In another aspect the tumour nucleic acid extracted from tumour cells shed from a solid tumour sample directly into the medium ex vivo may be combined with nucleic acid extracted from tumour cells released during mechanical disruption of at least part of the tumour sample. In one aspect the combined extracted nucleic acid may be sequenced.

### Cell Isolation and Nucleic Acid Extraction

In one aspect the method according to the present invention comprises a step of isolating the shed and/or released tumour cells as described herein from the medium.

The shed and/or released tumour cells may be isolated from the medium using methods known in the art. By way of example, the cells may be isolated using filtration and/or centrifugation as described in the present Examples.

In one aspect the method according to the present invention comprises a step of extracting nucleic acid from the shed and/or released tumour cells.

The nucleic acid according to the invention as described here may be DNA and/or RNA.

Nucleic acid, such as DNA and/or RNA suitable for downstream sequencing can be isolated from a sample using methods which are known in the art. For example DNA and/or RNA isolation may be performed using phenol-based extraction. Phenol-based reagents contain a combination of denaturants and RNase inhibitors for cell and tissue disruption and subsequent separation of DNA or RNA from contaminants. For example, extraction procedures such as those using DNAzol™, TRIZOL™ or TRI REAGENT™ may be used. DNA and/or RNA may further be isolated using solid phase extraction methods (e.g. spin columns) such as PureLink™ Genomic DNA Mini Kit or QIAGEN RNeasy™ methods. Isolated RNA may be converted to cDNA for downstream sequencing using methods which are known in the art (RT-PCR).

In one aspect the method of the invention comprises the following steps:

(a) providing a medium containing tumour cells shed from a solid tumour sample;

(b) isolating the shed tumour cells from the medium; and

(c) extracting nucleic acid from the shed tumour cells.

In one aspect the method of the invention comprises the following steps:

(a) providing a medium containing shed and/or released tumour cells as described herein;

(b) isolating the tumour cells from the medium; and

(c) extracting nucleic acid from the shed and/or released tumour cells.

In one aspect the number of tumour cells isolated from the medium is at least about 0.25×10⁵, 0.5×10⁵, 1×10⁵, 2×10⁵, 5×10⁵,1×10⁶, 5×10⁶, 10×10⁶, 15×10⁶, 20×10⁶, 25×10⁶, 30×10⁶, 35×10⁶, 40×10⁶, 45×10⁶ or 50×10⁶ cells.

In one aspect the number of tumour cells isolated from the medium is more than about 0.25×10⁵, 0.5×10⁵, 1×10⁵, 2×10⁵, 533 10⁵, 1×10⁶, 5×10⁶, 10×10⁶, 15×10⁶, 20×10⁶, 25×10⁶, 30×10⁶, 35×10⁶, 40×10⁶, 45×10⁶ or 50×10⁶ cells.

In one aspect the number of tumour cells isolated from the medium is about 10×10⁶ to 140×10⁶ cells per gram of tumour sample.

In one aspect the number of tumour cells isolated from the medium at least about 1×10⁶.

In one aspect the number of tumour cells isolated from the medium is more than about 1×10⁶.

In one aspect the number of tumour cells isolated from the medium at least about 1×10⁵.

In one aspect the number of tumour cells isolated from the medium is more than about 1×10⁵.

### Purification

Solid tumours are infiltrated by nucleated cells of non-tumour origin, including heterogeneous lymphocyte subpopulations, fibroblasts, and endothelial cells. The amount and composition of infiltrating cells is highly variable and patient dependent, which makes analysis of tumour samples difficult. Furthermore, the presence of contaminating cells leads to a reduction of sensitivity caused by measurement of irrelevant signals during sequencing, in some cases posing a significant risk to clonal neoantigen identification. As demonstrated in the present Examples, depletion of these unwanted cells improves the purity of samples, with higher tumour cell frequency, thus increasing the signal-to-noise ratio during nucleotide sequencing.

In one aspect of the present invention the method may comprise the step of removing non-tumour cells by negative selection, for example prior to the extraction of nucleic acid for sequencing.

In one aspect negative selection may comprise depletion of CD45+ cells, red blood cells, platelets, granulocytes, heterogeneous lymphocyte populations, fibroblasts, endothelial cells and/or hematopoietic cells.

Said negative selection may be carried out by immunomagnetic negative selection.

Immunomagnetic separation is a laboratory tool that can efficiently isolate cells from medium through the specific capture of biomolecules through the attachment of small magnetized particles or beads which are coated with antibodies specific for an antigen on the target cell.

Immunomagnetic separation typically involves coupling of biological macromolecules, such as specific antibodies, to superparamagnetic iron oxide (Fe₃O₄) particles.

Superparamagnetic particles exhibit magnetic properties when placed within a magnetic field but have no residual magnetism when removed from the magnetic field. This technology has been incorporated to make uniform porous polystyrene spheres, approximately 2-5 μm in diameter, with an even dispersion of magnetic Fe₃O₄ throughout the bead. These magnetic beads are coated with a thin polystyrene shell that encases the magnetic material and provides a defined chemical surface area for the adsorption of coupling of molecules such as antibodies.

The magnetic particles are added to a heterogeneous suspension to bind to the desired target (non-tumour cells according to the present invention) and form a complex composed of the magnetic particle and target. A magnet is used to immobilize the magnetic particles complexed with the target against the vessel wall, and the remainder of the material is removed. Washing steps are easily performed while the particle—target complex is retained.

In one aspect a commercially available kit may be used for negative selection, for example one or more of EasySep Direct Human CD45 depletion kit (StemCell #17898), EasySep Direct Human PBMC Isolation kit (StemCell # 19654), Tumour Cell Isolation Kit (Miltenyi Biotec # 130-108-339) and/or EasySep Direct Human Circulating Tumour Cell (CTC) Enrichment Kit (StemCell #19657), according to manufacturer's instructions.

In one aspect negative selection may comprise a first step of mononuclear cell isolation (for example using the PBMC Isolation kit), and a second step of CD45+ cell depletion. Such a method will deplete red blood cells, granulocytes and platelets in step 1 and hematopoietic cells in step 2.

In one aspect the EasySep Direct Human PBMC Isolation kit (StemCell # 19654) may be used to deplete red blood cells, platelets and granulocytes, followed by EasySep Direct Human CD45 depletion kit (StemCell #17898) to deplete hematopoietic cells.

In a further aspect, the Tumour Cell Isolation Kit (Miltenyi Biotec #130-108-339) may be sued to deplete red blood cells, heterogeneous lymphocyte populations, fibroblasts and endothelial cells.

In a further aspect, the EasySep Direct Human Circulating Tumour Cell (CTC) Enrichment Kit (StemCell #19657) may be used to deplete red blood cells, platelets and hematopoietic cells.

Such kits employ magnetic beads for cell separation. For example, StemCell kits may use either EasySep Direct RapidSpheres (PBMC Isolation and CTC Enrichment kits) or EasySep Dextran RapidSpheres (CD45 depletion). The Miltenyi kit uses MACS MicroBeads.

In an alternative aspect, said negative selection may be carried out using cell adhesion-based separation, or cell density or size-based separation (for example by density gradient centrifugation or filtration).

In a further alternative contaminating cells may be labelled and fluorochrome activated cell sorting may be used to remove these cells, retaining the negative, unlabelled fraction (tumour cells).

Cells may be assessed by flow cytometry using human lineage markers for known contaminating cells, for example, CD45 (clone H130, Biolegend #368528), CD31 (clone WM59, Biolegend #303122), CD235a (clone REA175, Miltenyi Biotec #130-120-474) and/or anti-Fibroblast marker (clone REA165, Miltenyi Biotec 130-100-136)]. In one aspect markers for different tumour cell types may also be assessed, for example non-small cell lung cancer CD326 (clone 9C4, Biolegend #324212) or melanoma tumour cells MCSP (clone 9.2.27, BD #562414) +MART-1 (clone EP1422Y, Abcam, #ab51061)+MCAM (clone EPR3208, Abcam #ab75769)], depending on tumour cell type.

### Sequencing

As described herein, the present invention provides nucleic acid that is suitable for sequencing.

As such, in one aspect of the invention the nucleic acid may be sequenced.

As discussed in detail below, tumour sequence information may be used to identify neoantigens, for example. Tumour sequencing and identification of neoantigens is useful for a variety of reasons, for example neoantigen identification has prognostic, diagnostic and therapeutic value for cancer patients. Neoantigen identification may have value in designing and refining a cancer patient's treatment plan.

In one aspect, the identification of neoantigens may facilitate the design and production of cancer therapies.

For example, neoantigens may be used to design cell therapies, such as T cell therapies as described in detail herein. Furthermore, neoantigens may be used in the generation of peptides for vaccination therapies, for example for a therapy predicated on vaccination against tumour neoantigens.

In addition, neoantigens may be used to isolate cells, such as T cells. For example, MHC complexes loaded with neoantigen may be used to isolate T cells from which the T cell receptors may be sequenced. Neoantigen identification could therefore be used for expanding cells, for isolating specific TCRs or to generate vaccines.

Sequencing as described herein may be carried out by any standard method known in the art, for example, Next Generation Sequencing (NGS), whole genome sequencing, RNA sequencing or whole-exome sequencing (WES).

### Clonal Neoantigen Identification

In one aspect of the invention as described herein, the sequence of the nucleic acid is used to identify a clonal neoantigen from the tumour. The present Examples demonstrate that the invention may provide sequence information that is suitable for the identification of clonal neoantigens.

A “neoantigen” is a tumour-specific antigen which arises as a consequence of a mutation within a cancer cell. Thus, a neoantigen is not expressed by healthy cells in a subject.

The neoantigen may be caused by any non-silent mutation which alters a protein expressed by a cancer cell compared to the non-mutated protein expressed by a wild-type, healthy cell.

A “mutation” refers to a difference in a nucleotide sequence (e.g. DNA or RNA) in a tumour cell compared to a healthy cell from the same individual. The difference in the nucleotide sequence can result in the expression of a protein which is not expressed by a healthy cell from the same individual.

For example, the mutation may be a single nucleotide variant (SNV), a multiple nucleotide variant (MNV), a deletion mutation, an insertion mutation, an indel mutation, a frameshift mutation, a translocation, a missense mutation or a splice site mutation resulting in a change in the amino acid sequence (coding mutation).

The mutations may be identified by exome sequencing, RNA-seq, whole genome sequencing and/or targeted gene panel sequencing and/or routine Sanger sequencing of single genes. Suitable methods are known in the art.

Descriptions of exome sequencing and RNA-seq are provided by Boa et al. (Cancer Informatics. 2014;13(Suppl 2):67-82.) and Ares et al. (Cold Spring Harb Protoc. 2014 Nov. 3;2014(11):1139-48); respectively. Descriptions of targeted gene panel sequencing can be found in, for example, Kammermeier et al. (J Med Genet. 2014 Nov; 51(11):748-55) and Yap KL et al. (Clin Cancer Res. 2014. 20:6605). See also Meyerson et al., Nat. Rev. Genetics, 2010 and Mardis, Annu Rev Anal Chem, 2013. Targeted gene sequencing panels are also commercially available (e.g. as summarised by Biocompare ((http://www.biocompare.com/Editorial-Articles/161194-Build-Your-Own-Gene-Panels-with-These-Custom-NGS-Targeting-Tools/)).

Sequence alignment to identify nucleotide differences (e.g. SNVs) in DNA and/or RNA from a tumour sample compared to DNA and/or RNA from a non-tumour sample may be performed using methods which are known in the art. For example, nucleotide differences compared to a reference sample may be performed using the method described by Koboldt et al. (Genome Res. 2012; 22: 568-576). The reference sample may be the germline DNA and/or RNA sequence.

In one aspect the neoantigen may be a clonal neoantigen.

A “clonal” neoantigen is a neoantigen arising from a clonal mutation. Clonal mutations are mutations which occur early in tumorigenesis and are encoded within essentially every tumour cell. A “subclonal” neoantigen is a neoantigen arising from a subclonal mutation, i.e a mutation occurring in a particular tumour cell later in tumourigenesis and found only in cells descended from that cell.

As such, a clonal neoantigen is a neoantigen which is expressed effectively throughout a tumour. A subclonal neoantigen is a neoantigen that which is expressed in a subset or a proportion of cells or regions in a tumour. ‘Expressed effectively throughout a tumour’ may mean that the clonal neoantigen is expressed in all regions of the tumour from which samples are analysed.

It will be appreciated that a determination that a mutation is ‘encoded within essentially every tumour cell’ refers to a statistical calculation and is therefore subject to statistical analysis and thresholds.

Likewise, a determination that a clonal neoantigen is ‘expressed effectively throughout a tumour’ refers to a statistical calculation and is therefore subject to statistical analysis and thresholds.

Various methods for determining whether a neoantigen is “clonal” are known in the art. Any suitable method may be used to identify a clonal neoantigen.

By way of example, the cancer cell fraction (CCF), describing the proportion of cancer cells that harbour a mutation, may be used to determine whether mutations are clonal or subclonal. For example, the cancer cell fraction may be determined by integrating variant allele frequencies with copy numbers and purity estimates as described by Landau et al. (Cell. 2013 Feb. 14;152(4):714-26).

Suitably, CCF values may be calculated for all mutations identified within each and every tumour region analysed. If only one region is used (i.e. only a single sample), only one set of CCF values will be obtained. This will provide information as to which mutations are present in all tumour cells within that tumour region and will thereby provide an indication if the mutation is clonal or subclonal.

A clonal mutation may be defined as a mutation which has a cancer cell fraction (CCF) 0.75, such as a CCF 0.80, 0.85. 0.90, 0.95 or 1.0. A subclonal mutation may be defined as a mutation which has a CCF <0.95, 0.90, 0.85, 0.80, or 0.75. In one aspect, a clonal mutation is defined as a mutation which has a CCF 0.95 and a subclonal mutation is defined as a mutation which has a CCF<0.95. In another aspect, a clonal mutation is defined as a mutation which has a CCF≥0.75 and a subclonal mutation is defined as a mutation which has a CCF<0.75.

As stated, determining a clonal mutation is subject to statistical analysis and threshold.

In one aspect a mutation may be defined as a clonal mutation if the 95% CCF confidence interval is >=0.75, i.e. the upper bound of the 95% confidence interval of the CCF is greater than or equal to 0.75.

In another aspect a mutation may be identified as clonal if there is more than a 50% chance or probability that its cancer cell fraction (CCF) reaches or exceeds the required value as defined above, for example 0.95, such as a chance or probability of 55%, 60%, 65%, 70%, 75%, 80%, 85%, 90%, 95% or more.

Probability values may be expressed as percentages or fractions. The probability may be defined as a posterior probability.

In one aspect, a mutation may be identified as clonal if there is more than a 50% chance that its cancer cell fraction (CCF) is ≥0.95.

In a further aspect, mutations may be classified as clonal or subclonal based on whether the posterior probability that their CCF exceeds 0.95 is greater or lesser than 0.5, respectively.

In another aspect a mutation may be identified as clonal if the probability that the mutation has a cancer cell fraction greater than 0.75 is ≥0.5.

In one aspect a clonal neoantigen is a neoantigen that is ubiquitous throughout the tumour. In one aspect the clonal neoantigen may be present in multiple regions of the tumour, such as in more than 1 region of the tumour, for example in 2, 3, 4, 5, 6, 7, 8, 9 or 10 regions of the tumour. The clonal neoantigen may be present in a multi-region sample set. In one aspect the clonal neoantigen may be identified in every region of the tumour sampled, i.e. it is ubiquitous in the tumour.

As described above, a clonal neoantigen is one which is encoded within essentially every tumour cell, that is the mutation encoding the neoantigen is present within essentially every tumour cell and is expressed effectively throughout the tumour. However, a clonal neoantigen may be predicted to be presented by an HLA molecule encoded by an HLA allele which is lost in at least part of a tumour. In this case, the clonal neoantigen may not actually be presented on essentially every tumour cell. As such, the presentation of the neoantigen may not be clonal, i.e. it is not presented within essentially every tumour cell. Methods for predicting loss of HLA are described in International Patent Publication No. WO2019/012296.

In one aspect of the invention as described herein the neoantigen is predicted to be presented within essentially every tumour cell (i.e. the presentation of the neoantigen is clonal).

### Neoantigen-Specific T Cell Therapy

As discussed herein, neoantigens may be a target for T cell therapy in the treatment of cancer. Neoantigens, such as clonal neoantigens, may be identified according to methods as described herein.

In one aspect the T cell therapy as described herein may comprise T cells which target a plurality i.e. more than one clonal neoantigen.

In one aspect the number of clonal neoantigens is 2-1000. For example, the number of clonal neoantigens may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, for example the number of clonal neoantigens may be from 2 to 100.

In one aspect, the T cell therapy as described herein may comprise a plurality or population, i.e. more than one, of T cells wherein the plurality of T cells comprises a T cell which recognises a clonal neoantigen and a T cell which recognises a different clonal neoantigen. As such, the T cell therapy comprises a plurality of T cells which recognise different clonal neoantigens.

In one aspect the number of clonal neoantigens recognised by the plurality of T cells is 2-1000. For example, the number of clonal neoantigens recognised may be 2, 3, 4, 5, 6, 7, 8, 9, 10, 20, 50, 100, 150, 200, 250, 300, 350, 400, 450, 500, 550, 600, 650, 700, 750, 800, 850, 900, 950 or 1000, for example the number of clonal neoantigens recognised may be from 2 to 100.

In one aspect the plurality of T cells recognises the same clonal neoantigen.

In one aspect the neoantigen may be a subclonal neoantigen as described herein.

In one aspect of the invention the method may comprise isolating tumour infiltrating lymphocytes (TIL) from at least part of the solid tumour sample. The TIL may be selectively expanded to produce a population of neoantigen-specific T cells.

In one aspect the present invention also provides a method for selectively expanding a T cell population for use in the treatment of cancer in a subject, where the T cell population comprises T cells which are specific for neoantigens, such as clonal neoantigens. During selective expansion, T cells that respond to one or more neoantigens are expanded in preference to other T cells in the starting material that do not respond to the neoantigen(s).

In one aspect said method may comprise the steps of:
(a) providing medium containing tumour cells shed and/or released from a solid tumour sample as described herein;
(b) extracting nucleic acid from the shed and/or released tumour cells as described herein;
(c) sequencing nucleic acid extracted from the shed and/or released tumour cells as described herein;
(d) identifying a neoantigen from the tumour using the sequence information obtained in step (c);
(e) isolating tumour infiltrating lymphocytes (TIL) from at least part of said solid tumour sample; and
(f) co-culturing the TIL with an antigen presenting cell which presents the neoantigen identified in step (d).

The method may also comprise a step of administering said expanded T cell population to a subject in need of treatment for cancer.

In one aspect is provided a method for treating cancer in a subject, wherein said method comprises:

(a) providing medium containing tumour cells shed and/or released from a solid tumour sample as described herein;

(b) extracting nucleic acid from the shed and/or released tumour cells as described herein;

(c) sequencing nucleic acid extracted from the shed and/or released tumour cells as described herein;

(d) identifying a neoantigen from the tumour using the sequence information obtained in step (c);
(e) isolating tumour infiltrating lymphocytes (TIL) from at least part of said solid tumour sample; and
(f) co-culturing the TIL with an antigen presenting cell which presents the neoantigen identified in step (d); and
(g) administering said TIL to said subject.

The antigen-presenting cells (APCs) may be artificial or irradiated APCs. In one aspect, the APCs are dendritic cells. The dendritic cells may be derived from monocytes obtained from the patient's blood, referred to herein as monocyte-derived dendritic cells (MoDCs).

In an alternative aspect, step (f) in the methods above may be carried out with an artificial MHC complex which is loaded with neoantigen peptide. The co-culturing step may be carried by any other suitable method known in the art, for example artificial presentation methods which result in the same cell expansion as antigen presenting cells.

In one aspect, the APCs may be pulsed with peptides which present the relevant neoantigen(s). The APCs may be pulsed with peptides containing the identified mutations as single stimulants or as pools of stimulating neoantigens or peptides. Alternatively, the APCs may be modified to express the neoantigen sequence(s), for example by transfecting the APCs with mRNA encoding the neoantigen sequence(s).

T cells may be isolated using methods which are well known in the art. For example, T cells may be purified from single cell suspensions generated from samples on the basis of expression of CD3, CD4 or CD8. T cells may be enriched from samples by passage through a Ficoll-paque gradient.

Expansion of T cells may be performed using methods which are known in the art. For example, T cells may be expanded by ex vivo culture in conditions which are known to provide mitogenic stimuli for T cells. By way of example, the T cells may be cultured with cytokines such as IL-2 or with mitogenic antibodies such as anti-CD3 and/or CD28.

Other suitable methods for said expansion will be known to those of skill in the art. For example, International Patent Publication No. WO2019/094642 describes a number of protocols for expansion of T cells in response to neoantigens.

The expanded T cell population may have an increased number of T cells that target one or more neoantigens. For example, the T cell population of the invention will have an increased number of T cells that target a neoantigen compared with the T cells in the sample isolated from the subject. That is to say, the T cell population will differ from that of a “native” T cell population (i.e. a population that has not undergone the identification and expansion steps discussed herein), in that the percentage or proportion of T cells that target a neoantigen will be increased, and the ratio of T cells in the population that target neoantigens to T cells that do not target neoantigens will be higher in favour of the T cells that target neoantigens.

The T cell population according to the invention may have at least about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100% T cells that target a neoantigen. For example, the T cell population may have about 0.2%-5%, 5%-10%, 10-20%, 20-30%, 30-40%, 40-50%, 50-70% or 70-100% T cells that target a neoantigen. In one aspect the T cell population has at least about 1, 2, 3, 4 or 5% T cells that target a neoantigen, for example at least about 2% or at least 2% T cells that target a neoantigen.

Alternatively put, the T cell population may have not more than about 5, 10, 15, 20, 25, 30, 35, 40, 45, 50, 55, 60, 65, 70, 75, 80, 85, 90, 91, 92, 93, 94, 95, 96, 97, 98, 99, 99.1, 99.2, 99.3, 99.4, 99.5, 99.6, 99.7, 99.8% T cells that do not target a neoantigen. For example, the T cell population may have not more than about 95%-99.8%, 90%-95%, 80-90%, 70-80%, 60-70%, 50-60%, 30-50% or 0-30% T cells that do not target a neoantigen. In one aspect the T cell population has not more than about 99, 98, 97, 96 or 95% T cells that do not target a neoantigen, for example not more than about 98% or 95% T cells that do not target a neoantigen.

An expanded population of neoantigen-reactive T cells may have a higher activity than a population of T cells not expanded, for example, using a neoantigen peptide. Reference to “activity” may represent the response of the T cell population to restimulation with a neoantigen peptide, e.g. a peptide corresponding to the peptide used for expansion, or a mix of neoantigen peptides. Suitable methods for assaying the response are known in the art. For example, cytokine production may be measured (e.g. IL2 or IFNγ production may be measured). The reference to a “higher activity” includes, for example, a 1-5, 5-10, 10-20, 20-50, 50-100, 100-500, 500-1000-fold increase in activity. In one aspect the activity may be more than 1000-fold higher.

The T cell population may be all or primarily composed of CD8+ T cells, or all or primarily composed of a mixture of CD8+ T cells and CD4+ T cells or all or primarily composed of CD4+ T cells.

The expanded T cell population as described herein may be used in vitro, ex vivo or in vivo, for example either for in situ treatment or for ex vivo treatment followed by the administration of the treated cells to the body.

The expanded T cell population may be reinfused into a subject. Suitable methods for generating, selecting, expanding and reinfusing T cells are known in the art.

The expanded T cell population may be administered to a subject at a suitable dose. The dosage regimen may be determined by the attending physician and clinical factors. It is accepted in the art that dosages for any one patient depend upon many factors, including the patient's size, body surface area, age, the particular compound to be administered, sex, time and route of administration, general health, and other drugs being administered concurrently.

The expanded T cell population dose may involve the transfer of a given number of T cells as described herein to a patient. The therapeutically effective amount of T cells may be at least about 10³ cells, at least about 10⁴ cells, at least about 10⁵ cells, at least about 10⁶ cells, at least about 10⁷ cells, at least about 10⁸ cells, at least about 10⁹cells, at least about 10¹⁰ cells, at least about 10¹¹ cells, at least about 10¹² or at least about 10¹³ cells.

In one aspect the invention provides an expanded T cell population obtained or obtainable by any of the methods as described herein. In one aspect the expanded T cell population may be used in therapy. In one aspect the expanded T cell population may be used in the treatment or prevention of cancer.

In one aspect is provided an expanded T cell population as described herein for use in the treatment or prevention of cancer.

In a further aspect is provided an expanded T cell population as described herein in the manufacture of a medicament for use in the treatment or prevention of cancer.

In a further aspect is provided a method of treating cancer in a subject comprising the steps of producing an expanded T cell population as described herein, and administering same expanded T cell population to said subject.

### Subject

In a preferred embodiment of the present invention, the subject is a mammal, preferably a cat, dog, horse, donkey, sheep, pig, goat, cow, mouse, rat, rabbit or guinea pig, but most preferably the subject is a human.

As defined herein “treatment” refers to reducing, alleviating or eliminating one or more symptoms of the disease which is being treated, relative to the symptoms prior to treatment.

“Prevention” (or prophylaxis) refers to delaying or preventing the onset of the symptoms of the disease. Prevention may be absolute (such that no disease occurs) or may be effective only in some individuals or for a limited amount of time.

Suitably, the cancer may be ovarian cancer, breast cancer, endometrial cancer, kidney cancer (renal cell), lung cancer (small cell, non-small cell and mesothelioma), brain cancer (gliomas, astrocytomas, glioblastomas), melanoma, merkel cell carcinoma, clear cell renal cell carcinoma (ccRCC), lymphoma, small bowel cancers (duodenal and jejunal), leukemia, pancreatic cancer, hepatobiliary tumours, germ cell cancers, prostate cancer, head and neck cancers, thyroid cancer and sarcomas.

In one aspect the cancer is selected from non-small cell lung cancer (NSCLC), melanoma, renal cancer, bladder cancer, head and neck cancer, and breast cancer.

In a preferred aspect the cancer is selected from NSCLC, melanoma and head and neck cancer.

### Combination Therapies

T cell therapies as described herein may also be combined with other suitable therapies, for example additional cancer therapies. In particular, the expanded T cell compositions or populations as described herein may be administered in combination with immune checkpoint intervention, co-stimulatory antibodies, chemotherapy and/or radiotherapy, targeted therapy or monoclonal antibody therapy.

Immune checkpoint molecules include both inhibitory and activatory molecules, and interventions may apply to either or both types of molecule. Immune checkpoint inhibitors include, but are not limited to, PD-1 inhibitors, PD-L1 inhibitors, Lag-3 inhibitors, Tim-3 inhibitors, TIGIT inhibitors, BTLA inhibitors and CTLA-4 inhibitors, for example. Co-stimulatory antibodies deliver positive signals through immune-regulatory receptors including but not limited to ICOS, CD137, CD27 OX-40 and GITR.

Examples of suitable immune checkpoint interventions which prevent, reduce or minimize the inhibition of immune cell activity include pembrolizumab, nivolumab, atezolizumab, durvalumab, avelumab, tremelimumab and ipilimumab.

A chemotherapeutic entity as used herein refers to an entity which is destructive to a cell, that is the entity reduces the viability of the cell. The chemotherapeutic entity may be a cytotoxic drug. A chemotherapeutic agent contemplated includes, without limitation, alkylating agents, anthracyclines, epothilones, nitrosoureas, ethylenimines/methylmelamine, alkyl sulfonates, alkylating agents, antimetabolites, pyrimidine analogs, epipodophylotoxins, enzymes such as L-asparaginase; biological response modifiers such as IFNα, IL-2, G-CSF and GM-CSF; platinum coordination complexes such as cisplatin, oxaliplatin and carboplatin, anthracenediones, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

‘In combination’ may refer to administration of the additional therapy before, at the same time as or after administration of the T cell composition according to the present invention.

In addition or as an alternative to the combination with checkpoint blockade, the T cell composition of the present invention may also be genetically modified to render them resistant to immune-checkpoints using gene-editing technologies including but not limited to TALEN and Crispr/Cas. Such methods are known in the art, see e.g. US20140120622. Gene editing technologies may be used to prevent the expression of immune checkpoints expressed by T cells including but not limited to PD-1, Lag-3, Tim-3, TIGIT, BTLA CTLA-4 and combinations of these. The T cell as discussed here may be modified by any of these methods.

The T cell according to the present invention may also be genetically modified to express molecules increasing homing into tumours and or to deliver inflammatory mediators into the tumour microenvironment, including but not limited to cytokines, soluble immune-regulatory receptors and/or ligands.

### Composition

The expanded T cell population as described herein may be provided in the form of a composition.

The composition may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Compositions, according to the current invention, are administered using any amount and by any route of administration effective for preventing or treating a subject. An effective amount refers to a sufficient amount of the composition to beneficially prevent or ameliorate the symptoms of the disease or condition.

The exact dosage is chosen by the individual physician in view of the patient to be treated. Dosage and administration are adjusted to provide sufficient levels of the active agent(s) or to maintain the desired effect in a subject. Additional factors which may be taken into account include the severity of the disease state, e.g., liver function, cancer progression, and/or intermediate or advanced stage of macular degeneration; age; weight; gender; diet, time; frequency of administration; route of administration; drug combinations; reaction sensitivities; level of immunosuppression; and tolerance/response to therapy. Long acting pharmaceutical compositions are administered, for example, hourly, twice hourly, every three to four hours, daily, twice daily, every three to four days, every week, or once every two weeks depending on half-life and clearance rate of the particular composition.

The active agents of the pharmaceutical compositions of embodiments of the invention are preferably formulated in dosage unit form for ease of administration and uniformity of dosage. The expression “dosage unit form” as used herein refers to a physically discrete unit of active agent appropriate for the patient to be treated. The total daily usage of the compositions of the present invention will be decided by the attending physician within the scope of sound medical judgment. For any active agent, the therapeutically effective dose is estimated initially either in cell culture assays or in animal models, potentially mice, pigs, goats, rabbits, sheep, primates, monkeys, dogs, camels, or high value animals. The cell-based, animal, and in vivo models provided herein are also used to achieve a desirable concentration, total dosing range, and route of administration. Such information is used to determine useful doses and routes for administration in humans.

A therapeutically effective dose refers to that amount of active agent that ameliorates the symptoms or condition or prevents progression of the disease or condition. Therapeutic efficacy and toxicity of active agents are determined by standard pharmaceutical procedures in cell cultures or experimental animals, e.g., ED 50 (dose therapeutically effective in 50% of the population) and LD 50 (dose lethal to 50% of the population). The dose ratio of toxic to therapeutic effects is the therapeutic index, which is expressed as the ratio, LD 50/ED 50. Pharmaceutical compositions having large therapeutic indices are preferred. The data obtained from cell culture assays and animal studies are used in formulating a range of dosage for human use.

As formulated with an appropriate pharmaceutically acceptable carrier in a desired dosage, the pharmaceutical composition or methods provided herein is administered to humans and other mammals for example topically for skin tumours (such as by powders, ointments, creams, or drops), orally, rectally, mucosally, sublingually, parenterally, intracisternally, intravaginally, intraperitoneally, intravenously, subcutaneously, bucally, sublingually, ocularly, or intranasally, depending on preventive or therapeutic objectives and the severity and nature of the cancer-related disorder or condition.

Injections of the pharmaceutical composition include intravenous, subcutaneous, intra-muscular, intraperitoneal, or intra-ocular injection into the inflamed or diseased area directly, for example, for esophageal, breast, brain, head and neck, and prostate inflammation.

Liquid dosage forms are, for example, but not limited to, intravenous, ocular, mucosal, pharmaceutically acceptable emulsions, microemulsions, solutions, suspensions, syrups, and elixirs. In addition to at least one active agent, the liquid dosage forms potentially contain inert diluents commonly used in the art such as, for example, water or other solvents; solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols, fatty acid esters of sorbitan, and mixtures thereof. Besides inert diluents, the ocular, oral, or other systemically-delivered compositions also include adjuvants such as wetting agents, emulsifying agents, and suspending agents.

Dosage forms for topical or transdermal administration of the pharmaceutical composition herein include ointments, pastes, creams, lotions, gels, powders, solutions, sprays, inhalants, or patches. The active agent is admixed under sterile conditions with a pharmaceutically acceptable carrier. Preservatives or buffers may be required. For example, ocular or cutaneous routes of administration are achieved with aqueous drops, a mist, an emulsion, or a cream. Administration is in a therapeutic or prophylactic form. Certain embodiments of the invention herein contain implantation devices, surgical devices, or products which contain disclosed compositions (e.g., gauze bandages or strips), and methods of making or using such devices or products. These devices may be coated with, impregnated with, bonded to or otherwise treated with the composition herein.

Transdermal patches have the added advantage of providing controlled delivery of the active ingredients to the eye and body. Such dosage forms can be made by dissolving or dispensing the compound in the proper medium. Absorption enhancers are used to increase the flux of the compound across the skin. Rate is controlled by either providing a rate controlling membrane or by dispersing the compound in a polymer matrix or gel.

Injectable preparations of the pharmaceutical composition, for example, sterile injectable aqueous or oleaginous suspensions are formulated according to the known art using suitable dispersing agents, wetting agents, and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or a suspending medium. For this purpose, bland fixed oil including synthetic mono-glycerides or di-glycerides is used. In addition, fatty acids such as oleic acid are used in the preparation of injectables. The injectable formulations are sterilized prior to use, for example, by filtration through a bacterial-retaining filter, by irradiation, or by incorporating sterilizing agents in the form of sterile solid compositions, which are dissolved or dispersed in sterile water or other sterile injectable medium. Slowing absorption of the agent from subcutaneous or intratumoral injection was observed to prolong the effect of an active agent. Delayed absorption of a parenterally administered active agent is accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release is controlled. Examples of other biodegradable polymers include poly (orthoesters) and poly(anhydrides). Depot injectable formulations are also prepared by entrapping the agent in liposomes or microemulsions that are compatible with body tissues.

Solid dosage forms for oral administration include capsules, tablets, pills, powders, and granules. In solid dosage forms, the active agent is mixed with at least one inert, pharmaceutically acceptable excipient or carrier such as sodium citrate, dicalcium phosphate, fillers, and/or extenders such as starches, sucrose, glucose, mannitol, and silicic acid; binders such as carboxymethylcellulose, alginates, gelatin, polyvinylpyrrolidinone, sucrose, and acacia; humectants such as glycerol; disintegrating agents such as agar-agar, calcium carbonate, potato or tapioca starch, alginic acid, certain silicates, and sodium carbonate; solution retarding agents such as paraffin; absorption accelerators such as quaternary ammonium compounds; wetting agents, for example, cetyl alcohol and glycerol monostearate; absorbents such as kaolin and bentonite clay; and lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using excipients such as milk sugar as well as high molecular weight PEG and the like. The solid dosage forms of tablets, dragees, capsules, pills, and granules are prepared with coatings and shells such as enteric coatings, release controlling coatings, and other coatings known in the art of pharmaceutical formulating. In these solid dosage forms, the active agent(s) are admixed with at least one inert diluent such as sucrose or starch. Such dosage forms also include, as is standard practice, additional substances other than inert diluents, e.g., tableting lubricants and other tableting aids such as magnesium stearate and microcrystalline cellulose. In the case of capsules, tablets and pills, the dosage forms may also include buffering agents. The composition optionally contains opacifying agents that release the active agent(s) only, preferably in a certain part of the intestinal tract, and optionally in a delayed manner. Examples of embedding compositions include polymeric substances and waxes.

### Kit

In one aspect the invention provides a kit comprising an expanded T cell population as described herein.

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure which can be had by reference to the specification as a whole. Accordingly, the terms defined immediately below are more fully defined by reference to the specification as a whole.

Amino acids are referred to herein using the name of the amino acid, the three-letter abbreviation or the single letter abbreviation.

The term “protein”, as used herein, includes proteins, polypeptides, and peptides.

Other definitions of terms may appear throughout the specification. Before the exemplary embodiments are described in more detail, it is to understand that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

### Further Aspects

The present invention further provides aspects as defined in the following numbered paragraphs (paras):

1. A method for obtaining tumour nucleic acid for sequencing, comprising providing a medium containing tumour cells shed from a solid tumour sample and extracting nucleic acid from the shed tumour cells.

2. A method for sequencing nucleic acid from a solid tumour, wherein said method comprises the steps of:
(i) providing a medium containing tumour cells shed from a sample of said solid tumour; and
(ii) extracting nucleic acid from the shed tumour cells.

3. The method according to para 1 or para 2 wherein neither said tumour sample nor said shed cells are cultured ex vivo prior to extraction of said nucleic acid.

4. The method according to any preceding para wherein there is no disruption of the solid tumour sample by non-mechanical means prior to nucleic acid extraction.

5. The method according to para 4 wherein there is no disruption of the solid tumour sample by enzymatic or other non-mechanical means prior to nucleic acid extraction.

6. The method according to any preceding para, wherein the method comprises the following steps:

(a) providing a medium containing tumour cells shed from a solid tumour sample;

(b) isolating the shed tumour cells from the medium; and

(c) extracting nucleic acid from the shed tumour cells.

7. The method according to any preceding para wherein the medium contains tumour cells which have been shed from the solid tumour sample directly into the medium ex vivo.

8. The method according to any preceding para wherein the solid tumour sample has been retained in the medium for a period of at least about 5 minutes, 10 minutes, 15 minutes, 30 minutes, 60 minutes/1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours or 12 hours or more prior to a step of extracting nucleic acid from the shed tumour cells.

9. The method according to any preceding para wherein the cells have been shed into the medium during storage or transport of the solid tumour sample.

10. The method according to any preceding para wherein the method does not comprise a step of mechanically disrupting the solid tumour sample prior to the extraction of nucleic acid for sequencing.

11. The method according to any one of paras 1 to 9 further comprising the step of mechanically disrupting at least part of the solid tumour sample and extracting nucleic acid from tumour cells released during the mechanical disruption.

12. A method for obtaining tumour nucleic acid for sequencing, comprising providing a medium containing tumour cells released from at least part of a tumour sample during mechanical disruption of the at least part of the tumour sample and extracting nucleic acid from said tumour cells.

13. A method for sequencing nucleic acid from a solid tumour, wherein said method comprises the steps of:
(i) providing a medium containing tumour cells released from at least part of a tumour sample during mechanical disruption of the at least part of the tumour sample; and
(ii) extracting nucleic acid from the released tumour cells.

14. The method according to para 12 or para 13 wherein neither said tumour sample nor said released cells are cultured ex vivo prior to extraction of said nucleic acid.

15. The method according to any one of paras 12 to 14 wherein there is no disruption of the solid tumour sample by non-mechanical means prior to nucleic acid extraction.

16. The method according to para 15 wherein there is no disruption of the solid tumour sample by enzymatic or other non-mechanical means prior to nucleic acid extraction.

17. The method according to any preceding para, wherein the method comprises the following steps:

(a) providing a medium containing tumour cells released from a solid tumour sample;

(b) isolating the released tumour cells from the medium; and

(c) extracting nucleic acid from said tumour cells.

18. The method according to any one of paras 12 to 17 wherein the medium contains tumour cells which have been released from the solid tumour sample directly into the medium ex vivo.

19. The method according to any one of paras 12 to 18 wherein the solid tumour sample has been retained in the medium for a period of at least about 5 minutes, 10 minutes, 15 minutes, 30 minutes, 60 minutes/1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours or 12 hours or more prior to a step of extracting nucleic acid from the released tumour cells.

20. A method for obtaining tumour nucleic acid for sequencing, comprising the steps of:

(i) providing a medium containing tumour cells shed from a solid tumour sample by a method according to any one of paras 1 to 11;

(ii) providing a medium containing tumour cells released from at least part of a tumour sample during mechanical disruption of the at least part of the tumour sample by a method according to any one of paras 12 to 20; and

(iii) extracting nucleic acid from said cells from (i) and (ii).

21. The method according to any preceding para wherein the solid tumour is selected from non-small cell lung cancer (NSCLC), melanoma, renal cancer, bladder cancer, head and neck cancer, and breast cancer.

22. The method according to any preceding para wherein the solid tumour is selected from NSCLC, melanoma and head and neck cancer.

23. The method according to any preceding para which further comprises the step of sequencing nucleic acid extracted from the shed and/or released tumour cells.

24. The method according to para 23 wherein the sequence information generated is suitable for the identification of a clonal neoantigen(s) from the tumour.

25. The method according to para 24 further comprising the step of identifying a clonal neoantigen(s) from the tumour.

26. The method according to any preceding para further comprising isolating tumour infiltrating lymphocytes (TIL) from at least part of the solid tumour sample.

27. The method according to para 27 wherein the TIL are selectively expanded to produce a population of clonal neoantigen-specific T cells (cNeT).

28. The method according to any preceding para further comprising the step of removing non-tumour cells by negative selection prior to the extraction of nucleic acid for sequencing.

29. The method according to para 28 wherein the negative selection comprises immunomagnetic negative selection.

30. The method according to para 29 wherein the immunomagnetic negative selection comprises depletion of CD45+cells, red blood cells, platelets, granulocytes, heterogeneous lymphocyte populations, fibroblasts, endothelial cells and/or hematopoietic cells.

31. The method according to any preceding para wherein the medium is selected from a group consisting of HypoThermosol, Dulbecco's Modified Eagle's Medium (DMEM), Ham's F10 medium, Ham's F12 medium, Advanced DMEM, Advanced DMEM/F12, minimal essential medium, DMEM/F-12, DMEM/F-15, Liebovitz L-15, RPMI 1640, Iscove's modified Dulbecco's media (IMDM), OPTI-MEM SFM, N2B27, MEF-CM, PBS or a combination thereof, wherein preferably the medium is HypoThermosol.

32. Use of tumour cells which have been shed from a solid tumour sample into a medium ex vivo to provide tumour nucleic acid for sequencing.

33. Use of tumour cells which have been released from at least part of a tumour sample during mechanical disruption of the at least part of the tumour sample to provide tumour nucleic acid for sequencing.

34. A method for selectively expanding a T cell population for use in the treatment of cancer in a subject, the method comprising the steps of:
(a) providing medium containing tumour cells shed from a solid tumour sample;
(b) extracting nucleic acid from the shed tumour cells;
(c) sequencing nucleic acid extracted from the shed tumour cells;
(d) identifying a neoantigen from the tumour using the sequence information obtained in step (c);
(e) isolating tumour infiltrating lymphocytes (TIL) from at least part of said solid tumour sample; and
(f) co-culturing the TIL with an antigen presenting cell which presents the neoantigen identified in step (d).

35. A method for selectively expanding a T cell population for use in the treatment of cancer in a subject, the method comprising the steps of:
(a) providing medium containing tumour cells released from at least part of a tumour sample during mechanical disruption of the at least part of the tumour sample;
(b) extracting nucleic acid from the released tumour cells;
(c) sequencing nucleic acid extracted from the released tumour cells;
(d) identifying a neoantigen from the tumour using the sequence information obtained in step (c);
(e) isolating tumour infiltrating lymphocytes (TIL) from at least part of said solid tumour sample; and
(f) co-culturing the TIL with an antigen presenting cell which presents the neoantigen identified in step (d).

The invention will now be described, by way of example only, with reference to the following Examples.

### EXAMPLES

### Methods

Tumour cell suspensions were obtained from either enzymatic (ED) or mechanic dissociation (MD) of tumour specimens. Tumour fragments were dissociated by enzymatic digestion using the Tumour Dissociation Kit, human (Miltenyi Biotec #130-095-929) components and the gentleMACS™ Octo Dissociator with Heaters (Miltenyi Biotec #130-096-427), prior to the enrichment procedure. Alternatively, tumour cell suspensions were obtained from the media where the tumour specimen was transported and mechanically processed (Hypothermosol FRS Preservation solution, Sigma #H4416). Both ED and MD cell suspensions were filtered through a 70 μm strainer (Falcon #352350) and cell counts (including red blood cells) were obtained using a haemocytometer. Viable cell count was performed with trypan blue staining, immediately before and after the enrichment process.

Cells were then pelleted by centrifugation at 450×g, room temperature, for 10 minutes, and resuspended in PBS+2% FCS+1 mM EDTA at the desired concentration. Cell suspensions were enriched by different immunomagnetic negative selection methods, according to manufacturer's instructions:
EasySep Direct Human PBMC Isolation kit (StemCell # 19654) to deplete red blood cells, platelets and granulocytes, followed by EasySep Direct Human CD45 depletion kit (StemCell #17898) to deplete hematopoietic cells.
Tumour Cell Isolation Kit (Miltenyi Biotec # 130-108-339) to deplete red blood cells, heterogeneous lymphocyte populations, fibroblasts and endothelial cells.
EasySep Direct Human Circulating Tumour Cell (CTC) Enrichment Kit (StemCell #19657) to deplete red blood cells, platelets and hematopoietic cells.

After separation, the purity of the enriched tumour cell suspension was assessed by flow cytometry using human lineage markers for known contaminating cells [CD45 (clone HI30, Biolegend #368528), CD31 (clone WM59, Biolegend #303122), CD235a (clone REA175, Miltenyi Biotec #130-120-474) and anti-Fibroblast marker (clone REA165, Miltenyi Biotec 130-100-136)], together with either non-small cell lung cancer [CD326 (clone 9C4, Biolegend #324212)] or melanoma tumour cells [MCSP (clone 9.2.27, BD #562414)+MART-1 (clone EP1422Y, Abcam, #ab51061)+MCAM (clone EPR3208, Abcam #ab75769)], depending on tumour cell type. A secondary AlexaFluor647-conjugated donkey anti-rabbit IgG antibody (Biolegend #406414) was used when required. Purified tumour cells were frozen for subsequent DNA extraction and whole exome sequencing.

### Example 1—Transport Medium (TM)

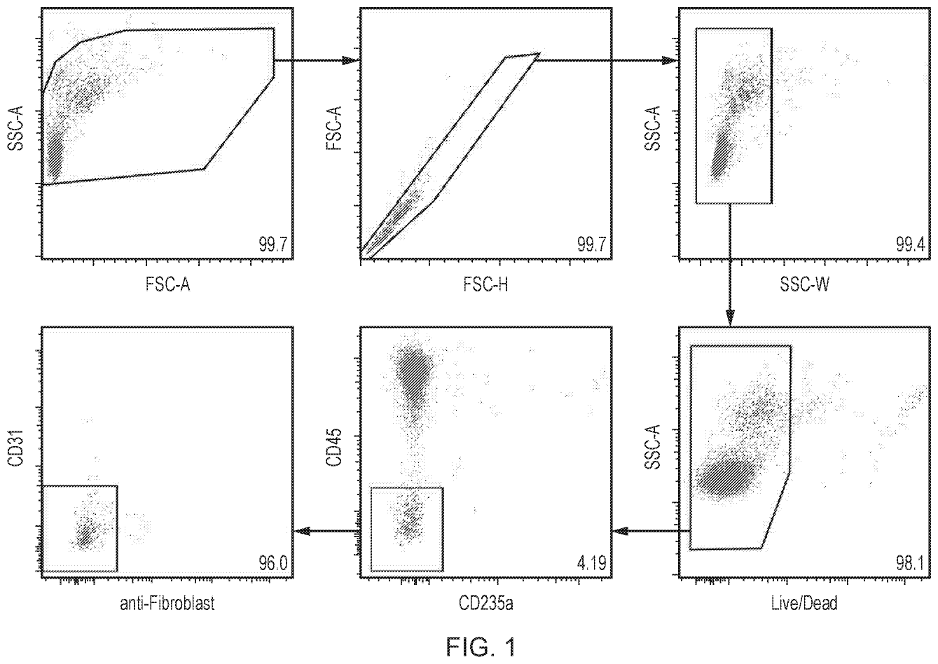

We firstly established a method to determine tumour cell frequency in a heterogeneous cell suspension by flow cytometry. Due to the variability of tumour cell markers' expression, the gating strategy consisted in sequential exclusion of known contaminating cells (FIG. 1A).

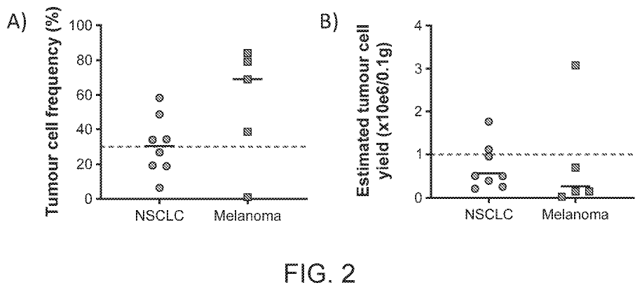

Although preparation of viable cell suspensions from enzymatically dissociated tumour specimens is possible, it is restricted by the amount of available tissue. Since tumour fragments are required for the isolation of tumour infiltrating lymphocytes (TILs), leftover fragments are typically around 0.1 g. Pondering the tumour cell frequency in cell suspensions obtained by enzymatic dissociation of small tumour fragments (FIG. 2A), it becomes obvious that the estimated tumour cell yield is on average below the threshold of 1 million cells (FIG. 2B), with a geometric mean of 0.565×10e6 (NSCLC) and 0.265×10e6 (Melanoma) tumour cells existing in a fragment of 0.1 g of tumour tissue. This highlights the necessity of using alternative sources of tumour cells.

The media where the tumour specimen is transported carries cells shed from the entire excised tumour, albeit mostly from its exterior surface. We explored this as an alternative source of tumour cells to use in analytical downstream applications, and so avoid the use of tumour specimens for this purpose.

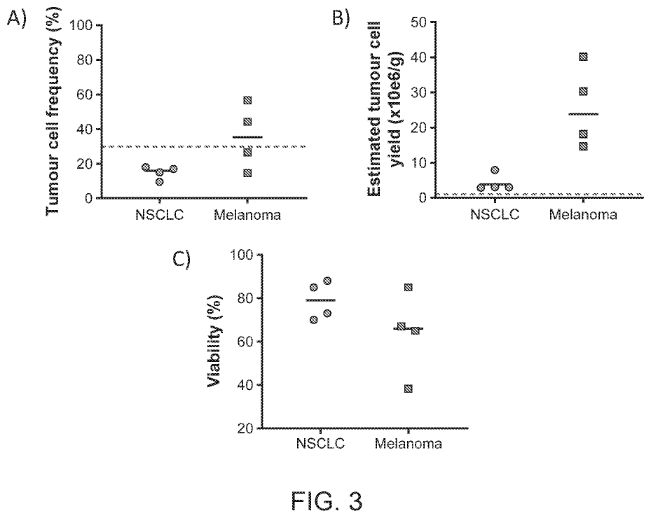

As a first observation, the tumour cell frequency in cell suspensions obtained from transport media is lower than in cell suspensions from enzymatically dissociated tumour fragments (FIGS. 2A and 3A). However, the total number of cells shed by the whole tumour is high and even when pondering the lower tumour cell frequency, the estimated tumour cell yield from transport media is comfortably above the 1 million cells threshold for both NSCLC and Melanoma samples (FIG. 3B). The geometric mean for each tumour type indicates that, in average, we can expect 3.834×10e6 (NSCLC) or 23.9×10e6 (Melanoma) tumour cells to be shed into the transport media, from each gram of tumour tissue. Importantly, cell viability is kept at reasonable levels for most samples, typically above 60% (FIG. 3C).

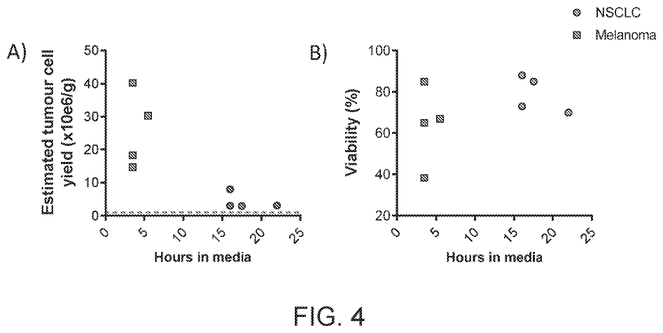

Tumours kept in the transport media for as little as 3.5 hours shed enough cells for use in downstream applications (FIG. 4A). So far, higher periods of time do not seem to have an effect in cell viability (FIG. 4B), but yield seems to be negatively affected.

### Example 2—Transport Medium and Cutting Medium (TM and CM)

The media where the tumour specimen is processed (Cutting media—CM) carries cells shed from the entire mass of excised tumour, both external and internally.

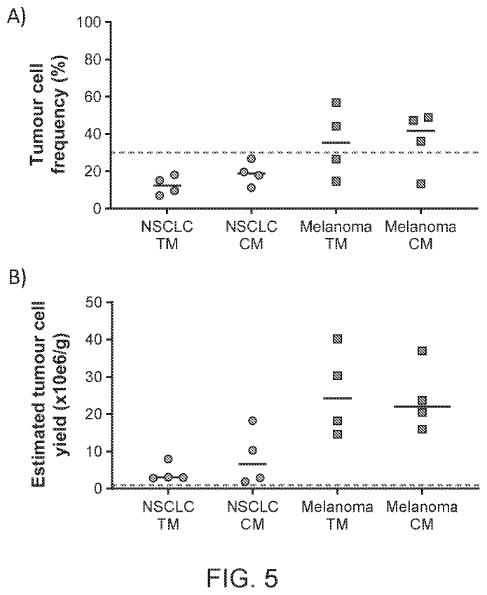

When compared, tumour cell frequency in either TM or CM is similar (FIG. 5A), as well as expected tumour cell yield (FIG. 5B). We can still see a considerable difference in estimated tumour cell yield between NSCLC and melanoma samples, even in CM.

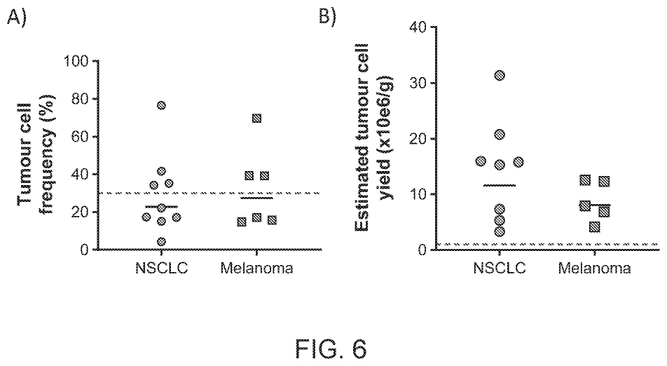

When pooling TM and CM together, we observed an overall improvement in the tumour cell frequency of NSCLC samples (FIG. 6). Interestingly there is a decrease in the number of melanoma tumour cells we can obtain by each gram of tumour tissue (in average, 8.06×10e6 cells per gram), as opposed to NSCLC samples (in average, 11.55×10e6 cells per gram). Direct comparisons between TM, CM and TM+CM must be done cautiously, as other variables were also shown to strongly affect yield, such as time of transport in the media.

### Example 3—Purification of TM and CM

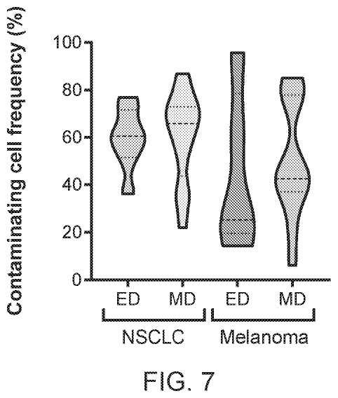

Using the gating strategy depicted in FIG. 1, we observed that the frequency of contaminating cells is highly variable and patient dependent (FIG. 7). The gating strategy depicted in FIG. 1 was also used to estimate the tumour cell frequency following depletion of unwanted cells, also allowing the calculation of recovery rate for each one of the tested kits.

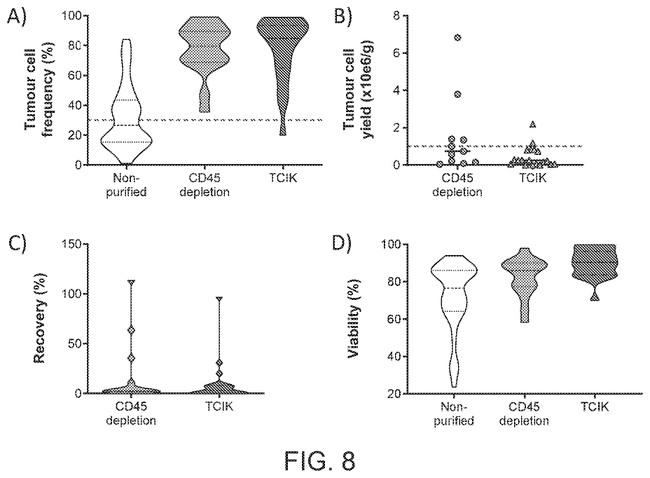

Both kits tested efficiently enriched the original cell suspension, with final tumour cell frequencies above the 30% threshold in most enriched samples (FIG. 8A). Tumour cell yield is disappointingly low for the two kits tested, with numbers falling under the 1 million cells threshold (FIG. 8B). This is explained by the extremely low recovery rates (FIG. 8C), and optimisation to circumvent this issue is under way. Cell viability was improved after enrichment, for both kits tested (FIG. 3C).

In summary, we have demonstrated that the media where the tumour is transported and processed is a reliable source for tumour cells. The resulting cell suspension can be enriched, yielding purity levels appropriate for sequencing and clonal neoantigen identification.

### Example 4—Sequencing of TM and CM Samples From NSCLC and Melanoma Tumours

Following purification, next generation sequencing was applied to the TM and CM samples. Firstly, the sequence data would provide an orthogonal validation of the improved tumour content following purification. Secondly, it was necessary to determine the ability to call somatic variation within the TM and CM samples.

DNA was extracted from the cell lysates obtained from both the purified and non-purified TM and CM samples using QIAmp DNA Mini kit, Cat. 51304. These samples underwent whole exome sequencing (WES) to a mean depth of 265x (range=224-302). Somatic variants were identified and the tumour content of the samples estimated using our proprietary PELEUS™ bioinformatics platform. The samples from Patient 1 originate from a NSCLC tumour, whilst samples from Patient 2 originate from a melanoma. Fresh frozen multi-region samples from the primary tumour of the matched patients had previously been sequenced (WES) to a mean depth of 220x along with a sample of the patient's blood (mean depth=92x) to be used as the germline control. These samples were also processed using the PELEUS™ platform and provide a ‘gold-standard’ comparison set for the TM and CM samples for the identification of clonal mutations.

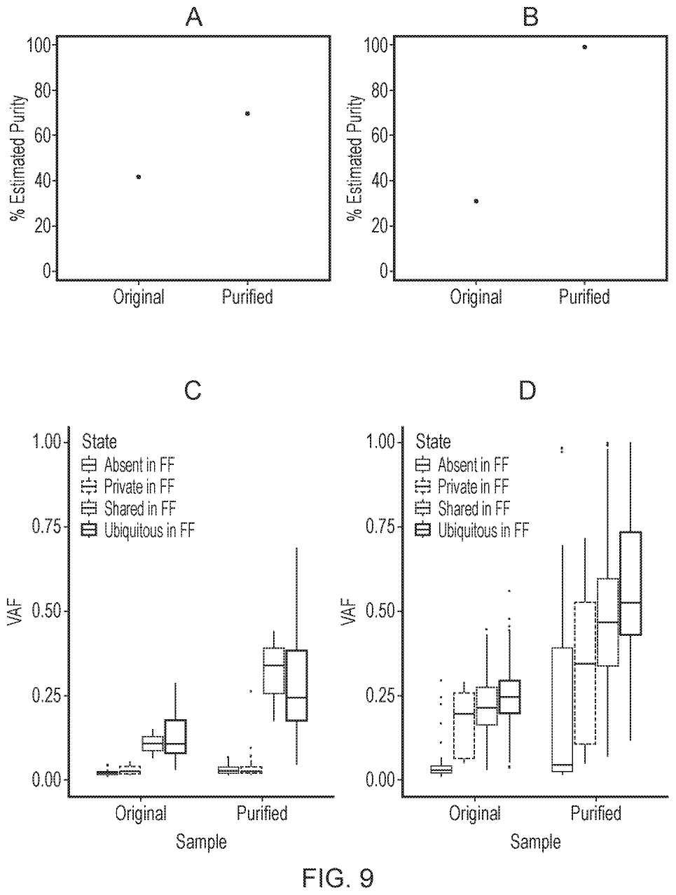

Tumour content estimates for the non-purified and purified TM and CM samples were calculated using the computational tool ASCAT (Van Loo et al. (PNAS, 2010, 107 (39): 16910-16915). In keeping with the estimates from flow cytometry, in both cases, the tumour content of the purified sample was found to be substantially higher than that of the non-purified sample (FIGS. 9A and B). This finding was further supported when comparing the variant allele frequencies (VAFs) of the somatic mutations identified in these samples (FIGS. 9C and D). The variants were classified according to their status in the primary multi-region dataset. A private mutation was only identified in a single multi-region sample. A shared mutation was identified in multiple samples from the same patient but not all samples. Finally, a ubiquitous mutation represents a mutation located in all primary regions obtained from the same patient and, for the purposes of this report, will be referred to as clonal mutations. Supportive of the utility of TM and CM samples for the accurate identification of somatic variation, mutations from the fresh frozen regions were re-identified and the VAFs of the mutations generally tallied with the class of mutation—with clonal mutations typically having a higher VAF. Additionally, the VAFs in the purified samples were higher than those in the non-purified samples, supporting the increased purity estimates.

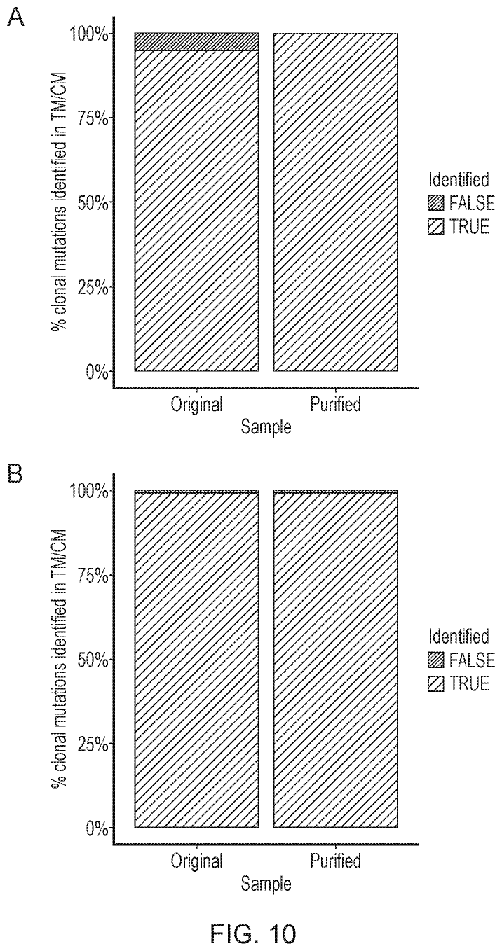

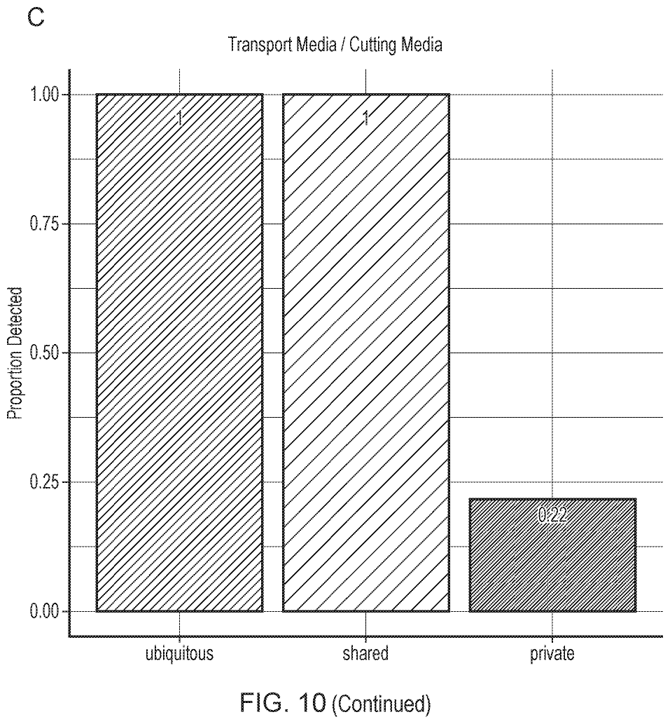

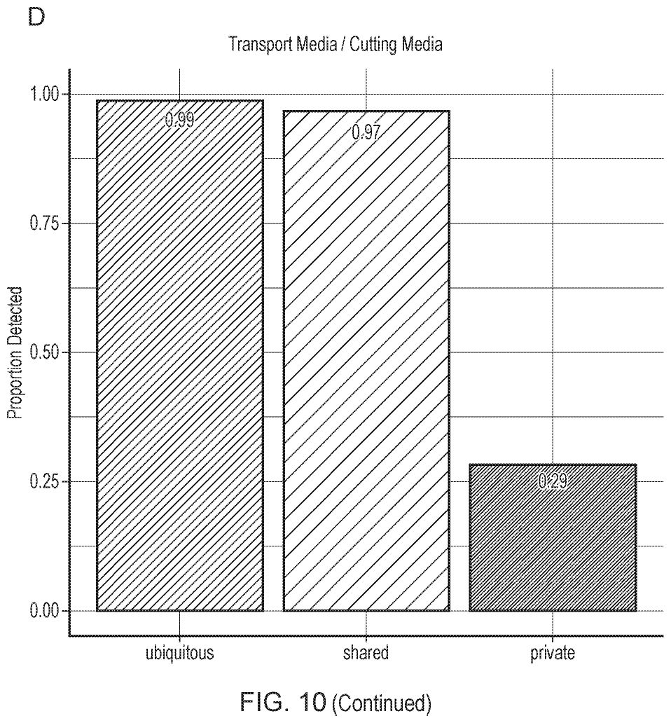

To determine the ability to detect clonal mutations in TM and CM samples, mutations from the multi-regional analysis were compared to the mutations identified in the TM and CM samples. For the non-purified samples, the positive percent agreement was >95% in both patients (FIG. 10). For the purified samples, the positive percent agreement was >99% in both patients. These results strongly support the applicability of TM and CM samples for the identification of clonal mutations.

### Example 5—Sequencing and Analysis of TM Sample

Following purification, next generation sequencing was applied to a TM sample to determine the ability to call somatic variation and re-identify annotated clonal mutations within the TM sample. This TM sample originated from a NSCLC tumour.

DNA was extracted from the cell lysate obtained from the purified TM sample. This sample underwent whole exome sequencing (WES) to a depth of 346x. Somatic single nucleotide variants were identified using our proprietary PELEUS™ bioinformatics platform. Fresh frozen multi-region samples from the primary tumour of the matched patient had previously been sequenced (WES) to a mean depth of 253x along with a sample of the patient's blood (depth=275x) to be used as the germline control. These samples were also processed using the PELEUS™ platform and provide a ‘gold-standard’ comparison set for the TM sample for the identification of clonal mutations.

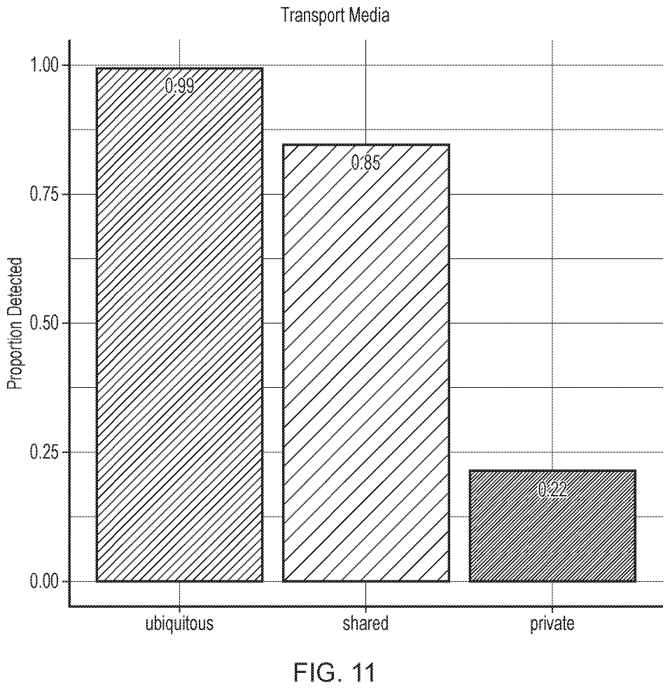

Variants were classified according to their status in the primary multi-region dataset. A private mutation was only identified in a single multi-region sample. A shared mutation was identified in multiple samples from the same patient but not all samples. Finally, a ubiquitous mutation represents a mutation located in all primary regions obtained from the same patient and, for the purposes of this report, will be referred to as clonal mutations. To determine the ability to detect mutations in TM samples, mutations from the multi-regional analysis were compared to the mutations identified in the TM sample (FIG. 11). The positive percent agreement (PPA) for detecting clonal mutations was 99% (179/180). The PPA for detecting shared mutations was 85% (71/84) and 22% (87/401) for detecting private mutations. These results strongly support the applicability of TM for the identification of clonal mutations.

### Example 6—Sequencing and Analysis of CM Sample

Following purification, next generation sequencing was applied to a CM sample derived from the same patient as in Example 5 to determine the ability to call somatic variation and re-identify annotated clonal mutations within the CM sample. This CM sample originated from a NSCLC tumour.

DNA was extracted from the cell lysate obtained from the purified CM sample. This sample underwent whole exome sequencing (WES) to a depth of 273x. Somatic single nucleotide variants were identified using our proprietary PELEUS™ bioinformatics platform. Fresh frozen multi-region samples from the primary tumour of the matched patient had previously been sequenced (WES) to a mean depth of 253x along with a sample of the patient's blood (depth=275x) to be used as the germline control. These samples were also processed using the PELEUS™ platform and provide a ‘gold-standard’ comparison set for the CM sample for the identification of clonal mutations.

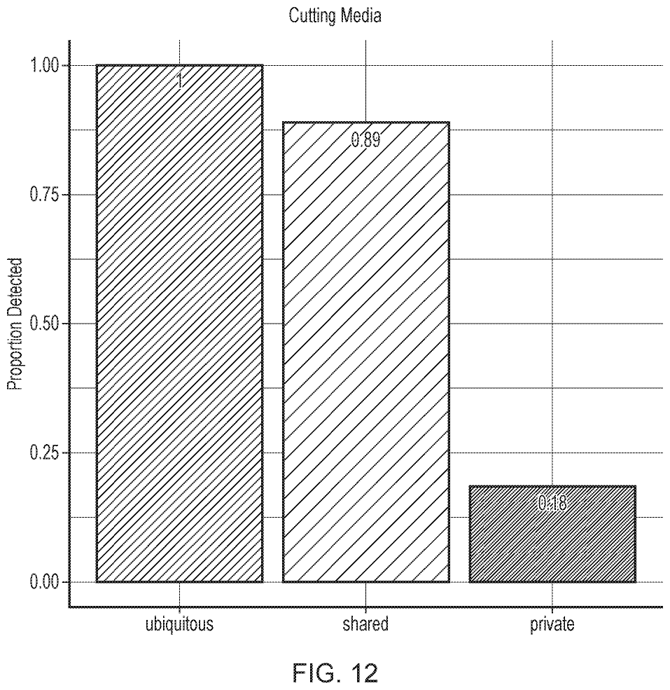

Variants were classified according to their status in the primary multi-region dataset. A private mutation was only identified in a single multi-region sample. A shared mutation was identified in multiple samples from the same patient but not all samples. Finally, a ubiquitous mutation represents a mutation located in all primary regions obtained from the same patient and, for the purposes of this report, will be referred to as clonal mutations. To determine the ability to detect mutations in CM samples, mutations from the multi-regional analysis were compared to the mutations identified in the CM sample (FIG. 12). The positive percent agreement (PPA) for detecting clonal mutations was 100% (180/180). The PPA for detecting shared mutations was 89% (75/84) and 18% (74/401) for detecting private mutations. These results strongly support the applicability of CM for the identification of clonal mutations.

### Example 7—Sequencing and Analysis of TM Sample Obtained from Head and Neck Squamous Cell Carcinoma

Following purification, next generation sequencing was applied to a TM sample derived from a Head and Neck Squamous Cell Carcinoma (HNSCC) tumour to determine the ability to call somatic variation and re-identify annotated clonal mutations within a TM sample from this cancer indication.

DNA was extracted and sequenced as described in Example 5 (the present sample underwent WES to a depth of 284x, the primary tumour of the matched patient had previously been sequenced (WES) to a mean depth of 364x and the sample of the patient's blood had been sequenced (WES to a depth 141x).

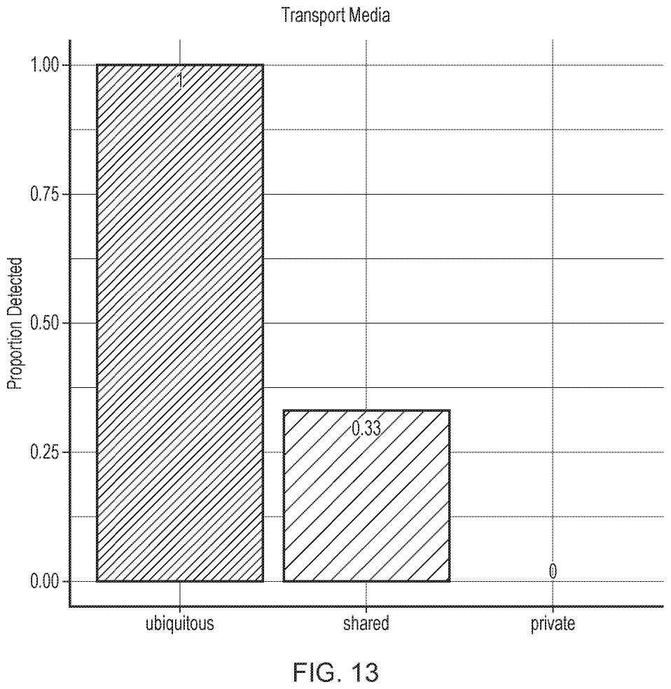

Variants were classified as described in Example 5. To determine the ability to detect mutations in TM samples, mutations from the multi-regional analysis were compared to the mutations identified in the TM sample (FIG. 13). The positive percent agreement (PPA) for detecting clonal mutations was 100% (147/147). The PPA for detecting shared mutations was 33% (7/21) and 0% (0/24) for detecting private mutations. These results strongly support the applicability of TM for the identification of clonal mutations.

### Example 8—Sequencing and Analysis of CM Sample Obtained from Head and Neck Squamous Cell Carcinoma

Following purification, next generation sequencing was applied to a CM sample derived from the same HNSCC tumour as used in Example 7. This was to determine the ability to call somatic variation and re-identify annotated clonal mutations within a CM sample from this cancer indication.

DNA was extracted and sequenced as described in Example 6 (the present sample underwent WES to a depth of 305x, the primary tumour of the matched patient had previously been sequenced (WES) to a mean depth of 364x and the sample of the patient's blood had been sequenced (WES to a depth 141x).

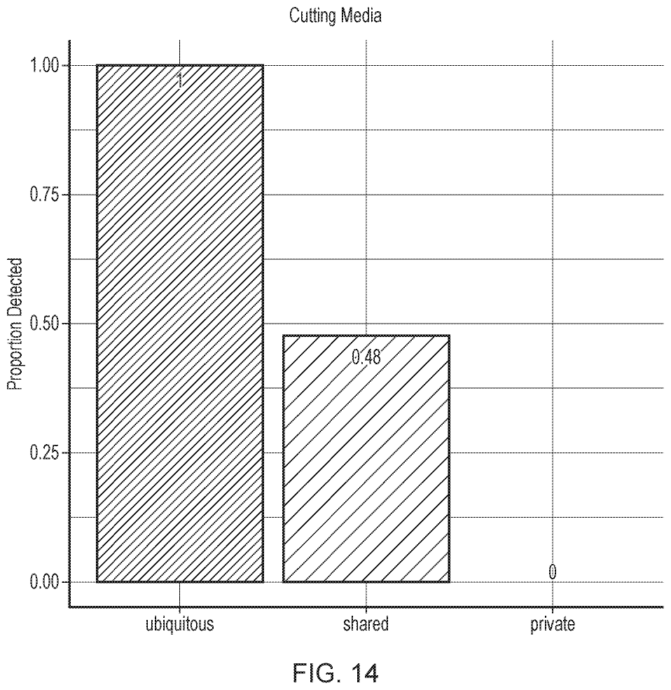

Variants were classified as described in Example 6. To determine the ability to detect mutations in CM samples, mutations from the multi-regional analysis were compared to the mutations identified in the CM sample (FIG. 14). The positive percent agreement (PPA) for detecting clonal mutations was 100% (147/147). The PPA for detecting shared mutations was 48% (10/21) and 0% (0/24) for detecting private mutations. These results strongly support the applicability of CM for the identification of clonal mutations.

1. A method for obtaining tumour nucleic acid for sequencing, comprising providing a medium containing tumour cells shed from a solid tumour sample and/or released during mechanical disruption of at least part of the solid tumour sample, and extracting nucleic acid from the shed and/or released tumour cells.

2. A method for sequencing nucleic acid from a solid tumour sample, wherein said method comprises the steps of:
(i) providing a medium containing tumour cells shed from said solid tumour sample and/or released during mechanical disruption of at least part of said tumour sample;
(ii) extracting nucleic acid from the shed and/or released tumour cells; and
(iii) sequencing said nucleic acid.

3. The method according to claim 1 wherein neither said tumour sample nor said shed or released tumour cells are cultured ex vivo prior to extraction of said nucleic acid.

4. The method according to claim 1, wherein there is no disruption of the solid tumour sample by enzymatic or other non-mechanical means prior to nucleic acid extraction.

5. The method according to claim 1, wherein the method comprises the following steps:
(a) providing a medium containing tumour cells shed from a solid tumour sample and/or released during mechanical disruption of at least part of the solid tumour sample;
(b) isolating the shed and/or released tumour cells from the medium; and
(c) extracting nucleic acid from the shed and/or released tumour cells.

6. The method according to claim 1 wherein the medium contains tumour cells which have been shed from the solid tumour sample directly into the medium ex vivo.

7. The method according to claim 1 wherein the solid tumour sample has been retained in the medium for a period of at least about or more prior to a step of extracting nucleic acid from the shed tumour cells.

8. The method according to claim 1, wherein the cells have been shed into the medium during storage or transport of the solid tumour sample.

9. The method according to claim 1, wherein the method does not comprise a step of mechanically disrupting the solid tumour sample prior to the extraction of nucleic acid for sequencing.

10. The method according to claim 1, wherein the solid tumour is selected from non-small cell lung cancer (NSCLC), melanoma, renal cancer, bladder cancer, head and neck cancer, and breast cancer.

11. (canceled)

12. The method according to claim 1, which further comprises the step of sequencing nucleic acid extracted from the shed and/or released tumour cells.

13. (canceled)

14. The method according to claim 12, further comprising the step of identifying a clonal neoantigen(s) from the tumour.

15. The method according to claim 1, further comprising isolating tumour infiltrating lymphocytes (TIL) from at least part of the solid tumour sample.

16. The method according to claim 15 wherein the TIL are selectively expanded to produce a population of clonal neoantigen-specific T cells (cNeT).

17. The method according to claim 1, further comprising the step of removing non-tumour cells by negative selection prior to the extraction of nucleic acid for sequencing.

18. The method according to claim 17 wherein the negative selection comprises immunomagnetic negative selection.

19. The method according to claim 18 wherein the immunomagnetic negative selection comprises depletion of CD45+ cells, red blood cells, platelets, granulocytes, heterogeneous lymphocyte populations, fibroblasts, endothelial cells and/or hematopoietic cells.

20. The method according to claim 1, wherein the medium is selected from a group consisting of HypoThermosol, Dulbecco's Modified Eagle's Medium (DMEM), Ham's F10 medium, Ham's F12 medium, Advanced DMEM, Advanced DMEM/F12, minimal essential medium, DMEM/F-12, DMEM/F-15, Liebovitz L-15, RPMI 1640, Iscove's modified Dulbecco's media (IMDM), OPTI-MEM SFM, N2B27, MEF-CM, PBS or a combination thereof.

21. (canceled)

22. A method for treating cancer in a subject, the method comprising selectively expanding a T cell population, wherein said method comprises the steps of:
(a) providing medium containing tumour cells shed from a solid tumour sample and/or released during mechanical disruption of at least part of the tumour sample;
(b) extracting nucleic acid from the shed and/or released tumour cells;
(c) sequencing nucleic acid extracted from the shed and/or released tumour cells;
(d) identifying a neoantigen from the tumour using the sequence information obtained in step (c);
(e) isolating tumour infiltrating lymphocytes (TIL) from at least part of said solid tumour sample;
(f) co-culturing the TIL with an antigen presenting cell which presents the neoantigen identified in step (d); and
(g) treating said subject with said expanded T cell population.
